(19) United States
(12) Patent Application Publication
(10) Pub. No.: US 2022/0016128 A1
(43) Pub. Date: Jan. 20, 2022

(54) METHODS, SYSTEM, AND KIT FOR MONITORING, DIAGNOSING, AND TREATING IMPULSIVE AGGRESSION

(71) Applicant: Supernus Pharmaceuticals, Inc., Rockville, MD (US)

(72) Inventors: Jennifer DUGAN STOCKS, Rockville, MD (US); Christopher EVANS, Boston, MA (US); Seung HWANG, Brooklyn, NY (US); Susan M. DALLABRIDA, Rowley, MA (US)

(73) Assignee: Supernus Pharmaceuticals, Inc., Rockville, MD (US)

(21) Appl. No.: 17/489,619

(22) Filed: Sep. 29, 2021

Related U.S. Application Data
(63) Continuation of application No. 16/851,980, filed on Apr. 17, 2020, now abandoned.
Application No. 16/851,980 is a division of application No. 15/499,357, filed on Apr. 27, 2017, now abandoned.
(60) Provisional application No. 62/329,688, filed on Apr. 29, 2016.

(51) Int. Cl. A61K 31/5377; G16H 50/20; G16H 50/30; G16H 10/20

(52) U.S. Cl. CPC A61K 31/5377; G16H 10/20; G16H 50/30; G16H 50/20

(57) ABSTRACT

The present disclosure relates to the treatment of impulsive aggression with molindone as an add-on therapy where patients are already receiving treatment for the underlying disease or disorder, e.g. attention deficit hyperactivity disorder (ADHD), bipolar disorder, autism, Tourette's syndrome or post traumatic stress disorder (PTSD), using evidence-based impulsive aggression testing methodologies and systems.

### CROSS-REFERENCE TO RELATED APPLICATIONS

The present application is a Continuation of U.S. application Ser. No. 16/851,980, filed Apr. 17, 2021, which is a Divisional of U.S. application Ser. No. 15/499,357, filed Apr. 27, 2017, which claims the benefit of U.S. Provisional Application No. 62/329,688, filed Apr. 29, 2016, the entire contents of which are incorporated herein by reference.

### BACKGROUND

Impulsive aggression, irritability, and similar syndromes represent a broad category of behaviors that complicate the management of several disease states, such as attention deficit hyperactivity disorder (ADHD), bipolar disorder, autism, Tourette's syndrome, and post traumatic stress disorder (PTSD). In some cases, 25-50% of patients optimally treated for the underlying disorder continue to manifest these syndromes (J Am Acad Child Adolesc Psychiatry, 2007 March; 46(3): (309-22).

The study of human aggression focuses on classifying aggressive behavior, which may be treated with either or both behavioral and pharmacological intervention. The present disclosure relates generally to evidence-based impulsive aggression monitoring and testing methodologies and systems. To the extent that methods of characterizing or monitoring impulsive aggression exist in the status quo, analysis thereof is difficult and do not always have predictive value for diagnosis, treatment, or evaluation of efficacy. Clinical decision-making for impulsive aggression patients is impeded by these issues.

Among potentially relevant interventions for impulsive aggression is molindone. See, e.g., U.S. Pat. No. 8,748,472, incorporated herein by reference.

Molindone is a weak base, exhibiting greater solubility in acidic to slightly acidic media than in neutral to slightly alkaline pH values (i.e., the physiologic pH range of the gastro-intestinal tract). As a weakly basic drug, molindone is typically included into formulations in the form of a salt, such as chloride, sulfate, phosphate, monohydrogenphosphate, dihydrogenphosphate, bromide, iodide, acetate, propionate, decanoate, caprylate, formate, oxalate, malonate, succinate, fumarate, maleate, citrate, lactate, tartrate, methanesulfonate, mandelate, and the like.

Molindone hydrochloride, a medium potency antipsychotic, was marketed as Moban® for the management of schizophrenia in adults. Moban is an immediate release (IR) tablet formulation provided at the dose strengths of 5 mg, 10 mg, 25 mg, 50 mg and 100 mg. As an IR dosage form it is taken 3 to 4 times daily with a typical maintenance dose range of 50 mg-100 mg per day. Limited molindone pharmacokinetic (PK) data is available in the literature. The drug substance has a reported bioavailability of 60%-70% relative to an intramuscular (IM) dose. It is absorbed rapidly following oral administration with a tₘₐₓ observed between 1 to 1.5 hours. The drug substance is extensively and rapidly metabolized with an oral dose plasma elimination half-life of about 2 hours.

Interventions such as molindone may be useful in the reduction of or the treatment of impulsive aggression, both by itself and as a secondary treatment where patients are already receiving treatment for the underlying disease or disorder, e.g. attention deficit hyperactivity disorder (ADHD), bipolar disorder, autism, Tourette's syndrome or post traumatic stress disorder (PTSD); however, there is a dearth in the art of appropriate metrics to evaluate their relevance due to the underlying lack of appropriate methods characterizing impulsive aggression.

Thus, further methodologies and systems for characterizing, monitoring, and evaluating impulsive aggression are required.

### SUMMARY OF DISCLOSURE

In some embodiments, the method includes treating a patient having impulsive aggression and who has been administered an impulsive aggression intervention, comprising:
(a) monitoring the patient for one or more aggressive episodes with a questionnaire to determine a post-intervention cumulative score; and either
(b) continuing to administer the impulsive aggression intervention if the post-intervention cumulative score is statistically different from a control cumulative score; or
(c) varying the impulsive aggression intervention if the post-intervention cumulative score is not statistically different from a control cumulative score;
wherein monitoring comprises calculating a cumulative score by a computer implemented algorithm from answers inputted into the questionnaire.

In some embodiments, the control cumulative score is a mean control cumulative score calculated by:
(i) monitoring each of a group of subjects who do not have impulsive aggression and have not been administered an impulsive aggression intervention for one or more aggressive episodes to determine each of the group of subjects' control cumulative score;
(ii) calculating the average of the control cumulative scores to determine a mean control cumulative score.

In some embodiments, each of the group of subjects who do not have impulsive aggression and have not been administered an impulsive aggression intervention have been administered a placebo intervention.

In some embodiments, the control cumulative score is calculated by:
(i) monitoring a subject who does not have impulsive aggression and has not been administered an impulsive aggression intervention for one or more aggressive episodes to determine a control cumulative score.

In some embodiments, each of subject who does not have impulsive aggression and has not been administered an impulsive aggression intervention has been administered a placebo intervention.

In some embodiments, the control cumulative score is a mean baseline cumulative score calculated by:
(i) monitoring each of a group of patients who have impulsive aggression and have not been administered an impulsive aggression intervention for one or more aggressive episodes to determine each of the group of patient's baseline cumulative score;
(ii) calculating the average of the baseline cumulative scores to determine a mean baseline cumulative score.

In some embodiments, each of the group of patients who have impulsive aggression and have not been administered an impulsive aggression intervention have been administered a placebo intervention.

In some embodiments, the control cumulative score is a baseline cumulative score calculated by monitoring the patient having impulsive aggression for one or more aggressive episodes prior to administration of the impulsive aggression intervention to determine a baseline cumulative score.

In some embodiments, the questionnaire comprises a diary used by a caregiver to report each of the one or more aggressive episodes after each of one or more aggressive episodes is observed. In some embodiments, the questionnaire further prompts the caregiver to review each of the one or more aggressive episodes reported for in a day and enter any aggressive episodes that were not previously reported.

In some embodiments, each of the one or more aggressive episodes is characterized by a group of behaviors comprising yelling, screaming, threatening, scratching, throwing, slamming, hitting self, arguing, cursing, name calling, shoving, hair pulling, fighting, hitting others, and kicking others; and wherein the behaviors are coded as observed (coded 1) or not observed (coded 0) based on answers from the caregiver. In some embodiments, a frequency of one or more behaviors over a fixed period of time is calculated to generate a score. In some embodiments, the fixed period of time is a week or seven days.

In some embodiments, the cumulative score is calculated based on two scores, each representing the frequency of a group of behaviors.

In some embodiments, the first score is calculated based on the frequency of a group of behaviors consisting of: yelling, screaming, threatening, scratching, throwing, slamming, and hitting self; and wherein the second score is calculated based on the frequency of a group of behaviors consisting of: arguing, cursing, name calling, shoving, hair pulling, fighting, hitting others, and kicking others. In some embodiments, the cumulative score is calculated as a weighted score. In some embodiments, the cumulative score is calculated as an unweighted score.

In some embodiments of the present method, the impulsive aggression intervention administered is selected from a plurality of impulsive aggression interventions that each has a different characteristic relative to each other. In some embodiments, the impulsive aggression intervention is administration of molindone according to a dose or a dose regimen. In particular embodiments, the dose of molindone is between about 12 mg and about 36 mg. In further particular embodiments, the dose of molindone is 18 mg. In further particular embodiments, the dose of molindone is 36 mg.

In some embodiments, continued administration of the impulsive aggression intervention comprises maintaining or lowering the dose of molindone.

In some embodiments, the patient is a child or an adolescent. In some embodiments, the child is between the ages of about 6 to 12. In some embodiments, the adolescent is between the ages of about 12 to 17.

In some embodiments, the patient with impulsive aggression has impulsive aggression associated with a primary condition. In some embodiments, the primary condition is attention deficit hyperactivity disorder (ADHD), bipolar disorder, autism, Tourette's syndrome or post traumatic stress disorder (PTSD).

A method of evaluating an impulsive aggression intervention for efficacy, comprising:
(a) monitoring a group of patients who have impulsive aggression and have been administered an impulsive aggression intervention for one or more aggressive episodes to determine each of the group of patient's post-intervention cumulative score;
(b) calculating the average of the baseline cumulative scores; and either
(c) determining the impulsive aggression intervention is efficacious if the post-intervention cumulative score is statistically different from a control cumulative score; or
(d) determining the impulsive aggression intervention is not efficacious if the post-intervention cumulative score is not statistically different from a control cumulative score,
wherein monitoring comprises calculating a cumulative score by a computer implemented algorithm from answers inputted into the questionnaire.

In some embodiments, the method includes continuing administration of the impulsive aggression intervention if the impulsive aggression intervention is determined to be efficacious.

In some embodiments, the control cumulative score is a mean control cumulative score calculated by:
(i) monitoring each of a group of patients who do not have impulsive aggression and have not been administered an impulsive aggression intervention for one or more aggressive episodes to determine each of the group of patients' control cumulative score;
(ii) calculating the average of the control cumulative scores to determine a mean control cumulative score.

In some embodiments, each of the group of subjects who do not have impulsive aggression and have not been administered an impulsive aggression intervention have been administered a placebo intervention.

In some embodiments, the control cumulative score is calculated by monitoring a subject who does not have impulsive aggression and has not been administered an impulsive aggression intervention for one or more aggressive episodes to determine a control cumulative score.

In some embodiments, each subject who does not have impulsive aggression and has not been administered an impulsive aggression intervention has been administered a placebo intervention.

In some embodiments, the control cumulative score is a mean baseline cumulative score calculated by:
(i) monitoring each of a second group of patients who have impulsive aggression and have not been administered an impulsive aggression intervention for one or more aggressive episodes to determine each of the group of patient's baseline cumulative score;
(ii) calculating the average of the baseline cumulative scores to determine a mean baseline cumulative score. In some embodiments, each of the second group of patients who have impulsive aggression and have not been administered an impulsive aggression intervention have been administered a placebo intervention.

In some embodiments, the control cumulative score is a baseline cumulative score calculated by:
(i) monitoring each of the group of patients who have impulsive aggression and have not been administered an impulsive aggression intervention for one or more aggressive episodes to determine each of the group of patient's baseline cumulative score;
(ii) calculating the average of the baseline cumulative scores to determine a mean baseline cumulative score. In some embodiments, the questionnaire comprises a diary used by a caregiver to report each of the one or more aggressive episodes after each of one or more aggressive episodes is observed.

In some embodiments, the questionnaire further prompts the caregiver to review each of the one or more aggressive episodes reported for in a day and enter any aggressive episodes that were not previously reported. In some embodiments, each of the one or more aggressive episodes is characterized by group of behaviors comprising yelling, screaming, threatening, scratching, throwing, slamming, hitting self, arguing, cursing, name calling, shoving, hair pulling, fighting, hitting others, and kicking others; and wherein the behaviors are coded as observed (coded 1) or not observed (coded 0) based on answers from the caregiver. In some embodiments, a frequency of one or more behaviors over a fixed period of time is calculated to generate a score. In some embodiments, the fixed period of time is a week or seven days. In some embodiments, the cumulative score is calculated based on two scores, each representing the frequency of a group of behaviors.

In some embodiments, the first score is calculated based on the frequency of a group of behaviors consisting of: yelling, screaming, threatening, scratching, throwing, slamming, and hitting self; and wherein the second score is calculated based on the frequency of a group of behaviors consisting of: arguing, cursing, name calling, shoving, hair pulling, fighting, hitting others, and kicking others. In some embodiments, the cumulative score is calculated as a weighted score. In some embodiments, the cumulative score is calculated as an unweighted score.

In some embodiments, the impulsive aggression intervention administered is selected from a plurality of impulsive aggression interventions that each has a different characteristic relative to each other. In some embodiments, the impulsive aggression intervention is administration of molindone according to a dose or a dose regimen. In some embodiments, the dose of molindone is between about 12 mg and about 36 mg. In particular embodiments, the dose of molindone is 18 mg. In particular embodiments, the dose of molindone is 36 mg. In further embodiments, continued administration of the impulsive aggression intervention comprises maintaining or lowering the dose of molindone.

In some embodiments, the patient is a child or an adolescent. In some embodiments, the child is between the ages of about 6 to 12. In some embodiments, the adolescent is between the ages of about 12 to 17.

In some embodiments, the patient with impulsive aggression has impulsive aggression associated with a primary condition. In some embodiments, the primary condition is attention deficit hyperactivity disorder (ADHD), bipolar disorder, autism, Tourette's syndrome or post traumatic stress disorder (PTSD).

In some embodiments, described herein is method of diagnosing impulsive aggression in a patient, comprising:
screening the patient for one or more aggressive episodes, wherein the screening comprises scoring a questionnaire, the scoring providing a cumulative score, and the screening further comprises determining the patient's aggressive episode risk based on the cumulative score from the answers to the questionnaire; and
choosing an impulsive aggression intervention if the cumulative score exceeds a threshold;
wherein the scoring of the questionnaire and the determination of the aggressive episode risk are performed using an algorithm implemented by a computer.

In some embodiments, the method further comprises administering to the patient an impulsive aggression intervention if the cumulative score exceeds a threshold.

In some embodiments, the further comprises:
administering to the patient the impulsive aggression intervention;
screening the patient for one or more aggressive episodes after the impulsive aggression intervention; and
determining if the impulsive aggression intervention is appropriate if the cumulative score is above or below the threshold.

In some embodiments, the questionnaire comprises a diary used by the caregiver to report each of the one or more aggressive episodes after each of one or more aggressive episodes is observed. In some embodiments, the questionnaire further prompts the caregiver to review each of the one or more aggressive episodes reported for the day and enter any aggressive episodes that were not previously reported. In some embodiments, each of the one or more aggressive episodes is characterized by group of behaviors comprising yelling, screaming, threatening, scratching, throwing, slamming, hitting self, arguing, cursing, name calling, shoving, hair pulling, fighting, hitting others, and kicking others; and wherein the behaviors are coded as observed (coded 1) or not observed (coded 0) based on answers from the caregiver. In some embodiments, a frequency of one or more behaviors over a fixed period of time is calculated to generate a cumulative score. In some embodiments, the cumulative score is calculated based on two scores, each representing the frequency of a group of behaviors. In some embodiments, the first score is calculated based on the frequency of a group of behaviors consisting of: yelling, screaming, threatening, scratching, throwing, slamming, and hitting self; and wherein the second score is calculated based on the frequency of a group of behaviors consisting of: arguing, cursing, name calling, shoving, hair pulling, fighting, hitting others, and kicking others. In some embodiments, the cumulative score is calculated as a weighted score.

In some embodiments, the cumulative score is calculated as an unweighted score. In some embodiments, the choosing step is performed by an algorithm.

In some embodiments, the impulsive aggression intervention is selected from a plurality of impulsive aggression interventions that each has a different characteristic relative to each other. In some embodiments, the impulsive aggression intervention is molindone.

In some embodiments, provided herein is a kit for use in a method comprising evaluating a patient for impulsive aggression and choosing an impulsive aggression intervention based on the screening, the kit comprising:
a screening tool that screens a patient for one or more aggressive episodes, wherein the screening comprises scoring a questionnaire and further comprising a screening score system based on a cumulative score from the answers to the questionnaire;
a diagnostic test that categorizes the aggressive episode risk based on the cumulative score from the questionnaire;
a suggested impulsive aggression intervention based on the patient's impulsive aggression score; and
instructions for implementing the suggested impulsive aggression intervention.

In some embodiments of the kit, the questionnaire comprises a diary used by the caregiver to report each of the one or more aggressive episodes after each of one or more aggressive episodes is observed. In some embodiments of the kit, the questionnaire further prompts the caregiver to review each of the one or more aggressive episodes reported for the day and enter any aggressive episodes that were not previously reported. In some embodiments of the kit, each of the one or more aggressive episodes is characterized by group of behaviors comprising yelling, screaming, threatening, scratching, throwing, slamming, hitting self, arguing, cursing, name calling, shoving, hair pulling, fighting, hitting others, and kicking others; and wherein the behaviors are coded as observed (coded 1) or not observed (coded 0) based on answers from the caregiver. In some embodiments of the kit, a frequency of one or more behaviors over a fixed period of time is calculated to generate a score. In some embodiments of the kit, the cumulative score is calculated based on two scores, each representing the frequency of a group of behaviors. In some embodiments of the kit, the first score is calculated based on the frequency of a group of behaviors consisting of: yelling, screaming, threatening, scratching, throwing, slamming, and hitting self; and wherein the second score is calculated based on the frequency of a group of behaviors consisting of: arguing, cursing, name calling, shoving, hair pulling, fighting, hitting others, and kicking others. In some embodiments of the kit, the cumulative score is calculated as a weighted score.

In some embodiments of the kit, the cumulative score is calculated as an unweighted score. In some embodiments of the kit, the impulsive aggression intervention is selected from a plurality of impulsive aggression intervention that each has a different characteristic relative to each other.

In some embodiments of the kit, the impulsive aggression intervention is molindone. In some embodiments of the kit, the screening tool comprises a graphical user interface that prompts the caregiver to respond to the questionnaire.

In some embodiments, provided is a method of evaluating a patient for impulsive aggression, the method comprising the steps of:
screening a patient for one or more aggressive episodes, wherein the screening comprises scoring a questionnaire, the scoring providing a cumulative score, and the screening further comprises determining the patient's aggressive episode risk based on the cumulative score from the answers to the questionnaire; and
determining the patient has impulsive aggression if the cumulative score exceeds a threshold;
administering to the patient the impulsive aggression intervention;
screening the patient for one or more aggressive episodes after the impulsive aggression intervention; and
determining if the impulsive aggression intervention is appropriate if the cumulative score is above or below the threshold
   wherein the scoring of the questionnaire and the determination of the aggressive episode risk are performed using an algorithm implemented by a computer.

In some embodiments, the method comprises continuing to administer the impulsive aggression intervention, if the impulsive aggression intervention is determined to be appropriate.

In some embodiments, the questionnaire comprises a diary used by the caregiver to report each of the one or more aggressive episodes after each of one or more aggressive episodes is observed. In some embodiments, the questionnaire further prompts the caregiver to review each of the one or more aggressive episodes reported for the day and enter any aggressive episodes that were not previously reported. In some embodiments, each of the one or more aggressive episodes is characterized by group of behaviors comprising yelling, screaming, threatening, scratching, throwing, slamming, hitting self, arguing, cursing, name calling, shoving, hair pulling, fighting, hitting others, and kicking others; and wherein the behaviors are coded as observed (coded 1) or not observed (coded 0) based on answers from the caregiver. In some embodiments, a frequency of one or more behaviors over a fixed period of time is calculated to generate a score. In some embodiments, the cumulative score is calculated based on two scores, each representing the frequency of a group of behaviors. In some embodiments, the first score is calculated based on the frequency of a group of behaviors consisting of: yelling, screaming, threatening, scratching, throwing, slamming, and hitting self; and wherein the second score is calculated based on the frequency of a group of behaviors consisting of: arguing, cursing, name calling, shoving, hair pulling, fighting, hitting others, and kicking others. In some embodiments, the cumulative score is calculated as a weighted score. In some embodiments, the cumulative score is calculated as an unweighted score. In some embodiments, the impulsive aggression intervention is selected from a plurality of impulsive aggression interventions that each has a different characteristic relative to each other. In some embodiments, the impulsive aggression intervention is molindone.

In some embodiments, provided herein is a computer configured for the input of data from a questionnaire used to monitor one or more patients for aggressive episodes with computer readable instructions, calculating a cumulative score and, optionally, one or more of the following processes selected from:
(a) treating a patient based on comparison to a control cumulative score;
(b) evaluating the efficacy of an impulsive aggression intervention;
(c) diagnosing impulsive aggression based on a threshold score;
(d) evaluating a patient for impulsive aggression based on a threshold score.

Aspects of the present disclosure provide methods and systems for monitoring, evaluating, diagnosing, or screening for impulsive aggression in patients. In some embodiments, this evaluation is based on caregiver input.

Such method aspects of the invention may comprise screening a patient for one or more aggressive episodes, wherein the screening comprises calculating a cumulative score based on answers inputted into a questionnaire, and the screening further comprises determining the patient's aggressive episode risk based on the cumulative score calculated from the answers to the questionnaire. In certain embodiments, any one or more of these steps is carried out by a computer implemented algorithm.

Further method aspects may comprise administering to the patient an impulsive aggression intervention; screening the patient for one or more aggressive episodes after the impulsive aggression intervention; and determining if the impulsive aggression intervention is appropriate if the cumulative score is above or below the threshold.

Still further method aspects relate to methods of evaluating the treatment of a patient having impulsive aggression and may comprise monitoring the patient for one or more aggressive episodes, wherein the monitoring comprises calculating a cumulative score based on answers inputted into a questionnaire. In some embodiments of the methods disclosed herein the patient may be administered or already have been administered an impulsive aggression intervention. Other method aspects relate to treating a patient having impulsive aggression by monitoring the patient, as described above, and either continuing or varying the treatment based on the comparison of a post-intervention cumulative score to a control cumulative score. For example, the impulsive aggression treatment may be continued if the post-intervention cumulative score is statistically different from a control cumulative score or varied if the post-intervention score is not statistically different from the control cumulative score.

Other method aspects contemplated herein involve methods of evaluating an impulsive aggression intervention and may comprise monitoring each of a group of patients for one or more aggressive episodes, wherein the monitoring comprises calculating a cumulative score based on answers inputted into a questionnaire. The cumulative scores may optionally be averaged to generate a mean post-intervention cumulative score. The mean post-intervention cumulative score may be compared to a control cumulative score to determine the efficacy of the impulsive aggression treatment. For example, the impulsive aggression intervention may be considered efficacious if there is a statistical difference from a control cumulative score or non-efficacious if there is no statistical difference from the control cumulative score.

In certain embodiments, the scoring of the questionnaire and/or the determination of the aggressive episode risk is performed using an algorithm implemented by a computer.

Still further aspects of the disclosure relate to method of evaluation of a patient or impulsive aggression intervention involving inputting patient data obtained from a questionnaire into a computer to determine cumulative scores and making treatment decisions or efficacy determinations such as those disclosed herein above.

In some embodiments, the questionnaire comprises a diary used by a caregiver to report each of the one or more aggressive episodes after each of one or more aggressive episodes is observed. In further embodiments, the questionnaire further prompts the caregiver to review each of the one or more aggressive episodes reported for the day and enter any aggressive episodes that were not previously reported.

In some embodiments, the one or more aggressive episodes are characterized by the behaviors exhibited by the patient. In embodiments relating to the questionnaire, a list of behaviors may be presented in the form of a checklist. In certain embodiments, this checklist may comprise, or alternatively consist essentially of, any one or more of the following 15 behaviors: yelling, screaming, threatening, scratching, throwing, slamming, hitting self, arguing, cursing, name calling, shoving, hair pulling, fighting, hitting others, and kicking others.

In some embodiments, the behaviors may be numerically coded based on whether or not they are observed; in further aspects, this coding may be binary—coding 1 for observed and coding 0 for not observed.

In certain embodiments, the coding values for one or more behaviors over a fixed period of time may be used to generate a score representing the frequency of the one or more behaviors over the fixed period of time. In some embodiments, the scores may represent the frequency of only one behavior. In some embodiments the scores may represent the frequency of a group of behaviors.

In some embodiments, this score may be used to calculate a cumulative score. In some embodiments, the cumulative score is calculated based on weighted scores. In some embodiments, the cumulative score is calculated based on unweighted scores.

Aspects of this disclosure contemplate that the cumulative score may be generated based on two scores, each representing the frequency of a group of behaviors. In certain embodiments, the first score is calculated based on the frequency of the following behaviors: yelling, screaming, threatening, scratching, throwing, slamming, and hitting self. In certain embodiments, the second score is calculated based the frequency of the following behaviors: arguing, cursing, name calling, shoving, hair pulling, fighting, hitting others, and kicking others.

Additional aspects of the disclosure relate to selecting an appropriate impulsive aggression intervention. In some aspects, the impulsive aggression intervention is chosen from a plurality of impulsive aggression intervention that each have a different characteristic relative to each other. In certain embodiments, the impulsive aggression intervention is molindone. In some embodiments the impulsive aggression intervention may be administration of molindone according to a dose or dose regimen. In some embodiments, the dose of molindone is between about 12 mg and 36 mg. In some embodiments, the dose of molindone is between about 18 mg and 36 mg. In some embodiments, the dose is lower than the dose of molindone administered for the treatment of schizophrenia.

In some aspects the impulsive aggression studied is associated with a primary condition, such as attention deficit hyperactivity disorder (ADHD), bipolar disorder, autism, Tourette's syndrome, or post traumatic stress disorder (PTSD).

Further aspects of the present disclosure relate to a kit comprising tools and instructions on carrying out the disclosed methods and a graphical user interface through which this evaluation may be conducted.

Certain aspects of the disclosure relate to computer implemented means and an interface for carrying out the above described methods. In some aspects, this includes computer readable instructions for instructing a processor to receive the information inputted into the questionnaire by the caregiver, computer readable instructions for instructing a processor to perform an algorithm using these data to determine an intervention commensurate in scope with the patient's impulsive aggression. Further such computer implemented means may involve a repetition of this process to re-evaluate the success or failure of the chosen intervention and computer readable instructions therefor.

### BRIEF DESCRIPTION OF THE DRAWINGS

FIG. 1A-J depict multiple screens of a graphical user interface for reporting aggressive episodes.

FIG. 2 is a flowchart depicting a non-limiting example of the disclosed methodology.

FIG. 3 depicts the correlation between cumulative scores calculated based on weekly averages of weighted scores and unweighted scores.

### DETAILED DESCRIPTION

It is to be understood that the present disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices and materials are now described. All technical and patent publications cited herein are incorporated herein by reference in their entirety. Nothing herein is to be construed as an admission that the present technology is not entitled to antedate such disclosure by virtue of prior invention.

### Definitions

As used in the specification and claims, the singular form “a”, “an”, and “the” include plural references unless the context clearly dictates otherwise.

“Aggressive episode” is used herein to refer to an event characterized by one or more aggressive behaviors.

As used herein, the term “caregiver” may refer to an adult, typically a parent or other caregiver who has the most daily contact with the patient. “Caregiver” can also refer to a spouse, significant other, or other relative who spends sufficient time with the patient to observe the patient's behavior. The term “caregiver” may also be used to intend a teacher, clinician, or supervisor capable of observing and reporting the patient's behavior.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

As used herein, the term “diary” refers to a record of one or more episodes over a fixed period of time.

The term “frequency” as used herein is a mathematical concept that refers to the number of times an event occurs calculated as a rate over a specified duration of time. In certain embodiments, frequency may be calculated over a day, a week, a month, or a year. In some embodiments, frequency may be calculated as an average value based on a number of frequencies—for example: frequency values may be obtained for each day of the week, added together, and divided by the days of the week to yield an average daily frequency for the week.

As used herein, “impulsive aggression” refers to repeated aggressive episodes related to an underlying primary disorder. In some embodiments, the underlying primary disorder is attention deficit hyperactivity disorder (ADHD), bipolar disorder, autism, Tourette's syndrome, or post traumatic stress disorder (PTSD).

As used herein, the term “intervention” can refer to any treatment, therapeutic, behavioral therapy, etc. that is used to mitigate, remedy, or resolve the symptoms or causes of impulsive aggression. Non-limiting examples include molindone and behavioral interventions.

The term “molindone” means 3-Ethyl-6,7-dihydro-2-methyl-5-(morpholinomethyl)indol-4(5H)-one or a pharmaceutically acceptable salt or ester thereof, including either a single (−) enantiomer, or in the form of a single (+) enantiomer, or in the form of a racemic mixture of both, or in the form of a non-racemic mixture of enantiomers with varying amounts of (−) and (+) enantiomers.

As used herein the term “observe” and variations thereof may be used in reference to a primary or secondary observation. A primary observation is one by a caregiver who directly observed the aggressive episode. A secondary observation is one reported to the caregiver by an outside source—e.g. another caregiver, an unaffiliated observer, or another patient.

The terms “monitoring,” “screening,” “evaluating,” and/or equivalents thereof are used herein refer to the analysis of the data input from the questionnaire, comprising calculating a cumulative score.

The term “computer” as used herein refers to an electronic device for storing and processing data, optionally in binary form, according to instructions given to it. The term “computer implemented algorithm” refers to the instructions given to the computer for the process of data analysis.

As used herein the term “statistical difference” refers to a statistically significant difference at an established alpha value, for example alpha=0.001, 0.01, or 0.05 based on a technique of statistical analysis. Statistical methods can be used to define the critical range of values. Typically within one standard deviation of those approximate values might be considered as statistically significant values for determining a statistically significant difference, preferably two standard deviations. For this reason, the word “about” is used in connection with the stated value. Certain statistical methods can be used to in the methods disclosed herein. These statistical methods may include, but are not limited to: 1) linear regression; 2) classification tree methods; and 3) statistical machine learning to optimize the unbiased performance of algorithms for making predictions. Each of these statistical methods is well-known to those of ordinary skill in the field of biostatistics and can be performed as a process in a computer. A large number of software products are available commercially to implement statistical methods, such as, without limitation, S-PLUS®, commercially available from Insightful Corporation of Seattle, Wash.

The terms “patient,” “subject,” and “individual” are as used interchangeably herein to refer to human subjects of any age group. In certain embodiments, the patients are children or adolescents. In further embodiments, the patients are between the ages of 6 to 12 years. In further embodiments, the patients are adolescents (12 to 17 years).

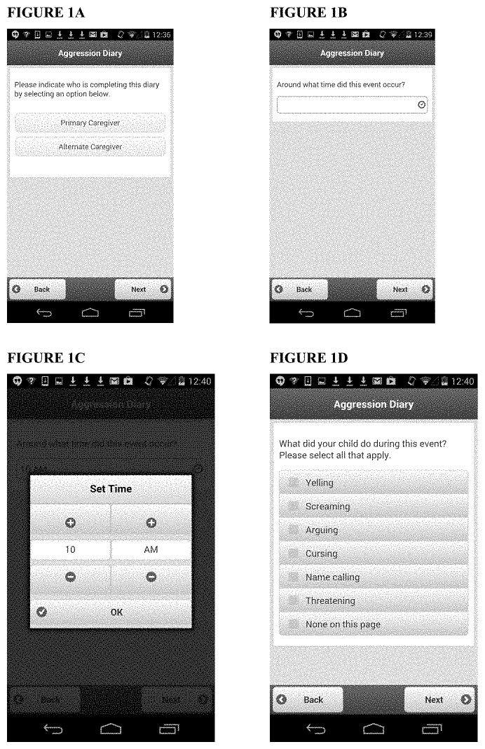

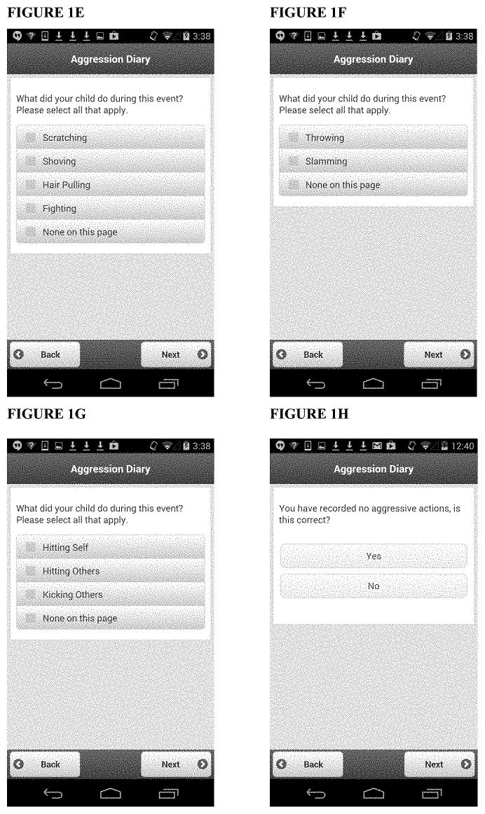

As used herein the term “questionnaire” refers to a series of questions asked to a caregiver. In some embodiments, these questions are presented on a graphical user interface (FIG. 1A-1J); in further embodiments, the input from this questionnaire is stored digitally and communicated to a computer for analysis of the data. In some embodiments, the questionnaire comprises additional questions geared at characterizing the aggressive episode, including but not limited to the time, place, caregiver identity, and nature of the observation.

As used herein, the term “score” refers to a numerical value representative of a specific behavior, set of behaviors, disease, disorder, or pathology. In some embodiments, scores are calculated based on the addition of counts representing observed behaviors or frequency thereof. In some embodiments, counts of one or more observed behavior are weighted based on their respective prevalence, generating a “weighted score”. In alternative embodiments, none of the counts are adjusted for prevalence, generating an “unweighted score.” The term “cumulative score” refers to scores generated through addition of multiple score values. A “control cumulative score” is used herein to refer to a score value used from which to assess statistical difference, as it is taken from a control patient or group. Such controls may be “baseline” cumulative scores taken from the same patient or a group of patients having impulsive aggression prior to administration of an impulsive aggression intervention; “mean” cumulative scores averaged from cumulative scores of patients having or not having impulsive aggression who have not been administered an impulsive aggression intervention; or cumulative scores of a single patient. In some embodiments, these control groups are paired based on the patient being assessed, evaluated, monitored, or screened.

As used herein, the term “threshold” is used to refer to a specific “cut-off” value such as a cumulative score that is determinative of the presence of impulsive aggression and/or a specific risk level of aggressive episode.

Modes of Practicing the Disclosure

The present disclosure provides methods and systems for monitoring, evaluating, diagnosing, or screening for impulsive aggression in patients. In some embodiments, this evaluation is based on caregiver input. Aspects of this disclosure provide for specific method steps wherein a caregiver is able to input observational information characterizing an aggressive episode into a questionnaire.

In some embodiments, this is done in the form of a diary over a fixed time period; i.e. input of such information can be carried out during or after one or more aggressive episodes, after the aggressive episode is reported to the caregiver, or at the end of the fixed time period upon reviewing the recorded aggressive episodes.

The information inputted into the questionnaire, in some embodiments, may be stored as data and used in a method for evaluating impulsive aggression as disclosed herein. In some embodiments, this method is computer implemented. In further embodiments, the questionnaire is accessed by the caregiver over an electronic means suited for data input such as a graphical user interface. In alternative embodiments, the questionnaire is accessed by the caregiver as a physical means suited for data input; non-limiting example of such means include but are not limited to blank paper, SCANTRON®, punch card, or any other such machine readable input form.

In certain embodiments, the information characterizing the aggressive episode comprises a group of behaviors comprising one or more of the following behaviors: yelling, screaming, threatening, scratching, throwing, slamming, hitting self, arguing, cursing, name calling, shoving, hair pulling, fighting, hitting others, kicking others, teasing, spitting, biting, weapons, ripping, breaking, vandalizing, destroying, fire setting, hitting animal, kicking self, severe injury to self, severe injury to others, severe injury to animals. In some embodiments, the group of behaviors comprises, or alternatively consists of, at least 5 of the listed behaviors, at least 10 of the listed behaviors, at least 15 of the listed behaviors, at most 15 of the listed behaviors, at most 20 of the listed behaviors, at most 30 of the listed behaviors, or all 31 of the listed behaviors.

In certain embodiments, the aggressive episode is characterized based on a group of 15 behaviors consisting of: yelling, screaming, threatening, scratching, throwing, slamming, hitting self, arguing, cursing, name calling, shoving, hair pulling, fighting, hitting others, and kicking others.

Additional information about the episode may also be documented.

In some embodiments, the questionnaire consists of a series of questions designed to elicit the following information:
(a) The identity of the caregiver (e.g. Who is the caregiver completing this diary?)
(b) The time of the incident (e.g. Around what time did this event occur?)
(c) Observed behaviors—optionally, in the form of a checklist (e.g. What did your child do during this event? Please select all that apply.) The list of behaviors may optionally be presented in more than one question. See, e.g. FIG. 1D-1G.
(d) The identity of the observer (e.g. How did you know about this event?)

In some embodiments, answers to these questions may prompt further questions; for example, if none of the aggressive behaviors are observed but an episode was reported, the questionnaire may include “You have recorded no aggressive actions, is that correct?” or another such confirmatory question. See, e.g. FIG. 1H.

In some embodiments, the information inputted into the questionnaire is stored as data. In some embodiments, this data may be recorded as a binary: 1 for observed behavior and 0 for not observed data. These data may optionally be utilized in a computer implemented means for calculating a cumulative score, which in turn may be used in the methods disclosed herein.

In some embodiments, the frequency of one or more of behaviors is assessed based on the input into the questionnaire. This frequency information can then be used to compute a cumulative score characterizing a patient's aggressive episode risk.

In some embodiments, the frequency of behaviors may be assessed independently or in a group and then incorporated into the cumulative score.

In some embodiments, frequency is determined as a cumulative frequency of one or more behaviors. In some embodiments, the individual frequencies may be weighted based on their overall prevalence and imputed importance in impulsive aggression pathology. In certain embodiments, this cumulative score is based on frequency of one or more behaviors characterizing an aggressive episode.

In some embodiments the determination of a cumulative score is generated based on an algorithm—a non-limiting example of which is a machine learning algorithm that analyzes the inputted data.

The cumulative scores calculated from this information are useful in a number of impulsive aggression (IA) related fields, particularly diagnosing, treating, and assessing efficacy of treatment of IA.

In some embodiments, a certain threshold score may be determined for behavior that constitutes impulsive aggression. Patients with a score above this threshold may be diagnosed with IA, while those that have a score below the threshold are considered to not have IA. In further embodiments, an appropriate IA intervention selected for patients that have scores indicative of IA.

In some embodiments, a threshold value or control cumulative score can be used as a baseline for evaluating the efficacy of an IA intervention. In such embodiments, patients may be assessed for IA, administered the IA intervention, and then monitored for changes in aggressive episode risk score; alternatively, the patient may already have received or be currently receiving an IA intervention and is monitored for changes in aggressive episode risk score. In some embodiments, the threshold value or control cumulative score may be the patient's score at the time the treatment or evaluation is begun—a baseline cumulative score; in some embodiments, the threshold value may be the diagnostic threshold value for IA described above. In some embodiments, the threshold value or control cumulative score may be generated according to the same means as the patient's cumulative score. For example, a control cumulative score may be obtained from a single patient not having impulsive aggression; a mean control or baseline score may be obtained from a group of patients having or not having impulsive aggression, respectively. Like a baseline cumulative score, such scores would be calculated for a patient or patients who have not been administered the IA intervention and who have, optionally, been administered a placebo.

A decrease in score of a certain magnitude or below the established threshold value may indicate successful treatment. Further, the effects of a specific intervention, dose, concentration, timing, mode of administration, etc. may all be evaluated based on fluctuations in cumulative score. Such fluctuations may be assessed based on statistical differences from a control value.

In similar embodiments, one or more index scores may be used to select the appropriate IA intervention; e.g., threshold score values may be established for each known IA intervention to form an index of scores associated with treatment. Patients with a score corresponding to a particular indexed score will be administered the associated treatment.

In some embodiments, any of the above disclosed evaluation steps may be computer implemented and/or assessed through statistical methods appropriate to determine significance.

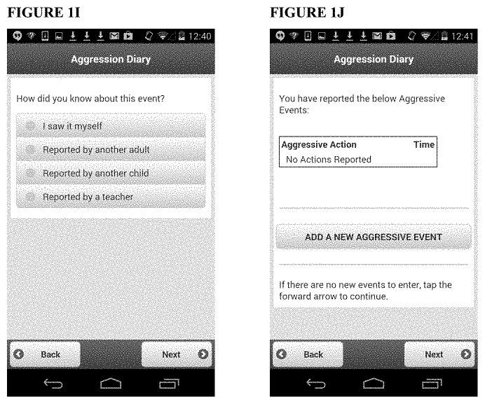

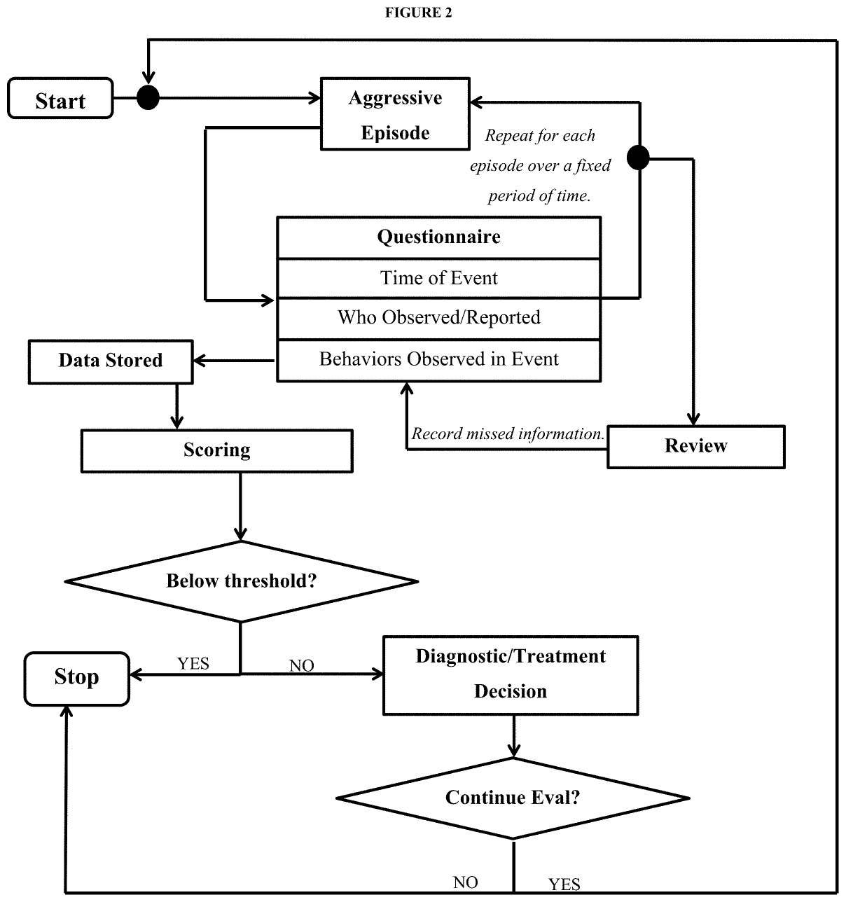

In certain embodiments, the described method is carried out according to the flow chart of FIG. 2:
An aggressive episode is observed by or reported to a caregiver.
The caregiver then inputs information about the episode into a questionnaire—specifically, the time, the observer of the event, and the behaviors observed.
Data is inputted over a fixed period of time, generally one day, at which point the caregiver is prompted to review the listed events.
Missed episodes may be inputted and changes to the previous data may be made during the prompted review period.
The data aggregated over one or more of the fixed time periods are scored, optionally using computed implemented means.
The computed cumulative score is compared to a threshold value to determine one or more of the following:
   if a patient has IA
   if a patient requires an IA intervention, and optionally which intervention is appropriate
   if a patient requires a change to his/her IA intervention
   if a patient requires continued evaluation
   if the patient's IA intervention was successful
The evaluation is, optionally, continued to monitor the patient's IA status or assess the intervention, according to one or more of the methods contemplated herein.

### Example 1: Psychometric Validity of Impulsive Aggression Evaluation Method

### Protocols for Instrument Testing

Psychometric properties of the IA evaluation questionnaire were assessed in a multicenter, non-interventional study in parents of pediatric male and female subjects (6 to 12 years of age, inclusive) with impulsive aggression comorbid with Attention Deficit Hyperactivity Disorder (ADHD) from Mar. 27, 2014 to Sep. 5, 2014. All study documents (Study Manual (serving as the protocol), Informed Consent Form (ICF), Case Report Form (CRF), and Demographic and Health Information Form (DHIF)) were reviewed and approved by Copernicus/Western independent review board (IRB) on Mar. 27, 2014 before subjects were screened for entry.

The objective of the validation study was to psychometrically validate the IA evaluation questionnaire through the analysis of:
Inter-item correlations—Estimated by tetrachoric correlations
Factor structure—Estimated by generalized linear exploratory factor analysis (EFA) model. Model fit indices were used to determine the number of factors to estimate. Oblique varimax rotated loading patterns were used to determine the factor structure of the IA evaluation questionnaire.
Item response theory (IRT)—Two item IRT models were considered for the binary IA evaluation questionnaire items: the 2 parametric logistic (2PL) IRT model and the Rasch or 1 parameter logistic (1PL) IRT model. These models differ only in how many slopes are estimated, with the 2PL estimating 1 slope per item and the Rasch/1PL not estimating a slope per item. Model fit indices were used to select the appropriate IRT model for the IA evaluation questionnaire. The factor structure of the IRT models was based on that obtained from the EFA. To support the construction of a single score in the context where subdomains are found to exist in EFAs, a Bi-Factor parameterization for the IRT model was explored. A Bi-Factor structure is one that augments an EFA subdomain structure with a general factor on which all items load. The Bi-Factor parameterization was compared to the EFA factor structure, and model fit indices were used to select the best model for the final solution.
Weighted and Unweighted scores were generated. Weighted scores were generated using the Bock and Mislevy empirical Bayes score method. See Bock, R. D. & Mislevy, R. J. Adaptive EAP Estimation of Ability in a Microcomputer Environment. Applied Psychological Measurement 1982; 6: 431-444, incorporated herein by reference. Unweighted scores were generated as the sum of the reported behaviors in each diary. While weighted scores are z-scores, the unweighted scores are count distributed.
Internal consistency reliability—Estimated by Kuder-Richardson eq. 20 (KR-20), and marginal reliability from IRT. KR-20 is the analog of Cronbach's alpha for binary items and marginal reliability is the IRT model-based analog for Cronbach's alpha.
Test-retest reliability—Estimated from intra-class correlation (ICC) for weighted scores and Spearman correlation for unweighted scores.
Concurrent validity—Estimated by Spearman correlation between IA evaluation questionnaire scores and both the Revised-Modified Overt Aggression Scale (R-MOAS) and Nisonger Child Behavior Rating Form—Typical IQ Disruptive Behavior (NCBRF-TIQ D-total scores). See Coccaro E F, Berman M E, Kavoussi R J: Assessment of life-history of aggression: development and psychometric characteristics. Psychiatry Res 1997; 73:147-157 and Shrout P E, Fleiss, J L. Intraclass Correlations: Use in Assessing Rater Reliability. Psychological Bulletin. 1979; 86(2):420-428, incorporated herein by reference.
Known-groups methods—Estimated at Day 14 by point and interval estimates of the IA evaluation questionnaire scores conditioned on the R-MOAS clinically significant aggression categories (score of 24+) and NCBRF-TIQ D-total problem behavior categories (85^{th} percentile score of 48+) using the appropriate linear or generalized linear model to which the IA evaluation questionnaire scores conform.
Minimal Clinical Important Difference (MCID)—Estimated via distribution-based methods derived using the SEM. Anchor-based MCID estimates were obtained by conditioning weighted and unweighted scores on caregiver impression of change (CGIC) strata.

Parents had to meet similar inclusion/exclusion criteria as in the development phase of the diary (i.e., concept elicitation and cognitive interviews) with the additional requirement that parents were willing to complete a diary for 14 days. One hundred subjects were targeted for enrollment across five sites in the United States (Bradenton, Fla., Maitland, Fla., Columbus, Ohio, Baltimore, Md., and Owensboro, Ky.) with actual enrollment at the end of the study being 103 subjects (n=103). The sample comprised mostly parents of male (70.9%) children, who were on average 8.7 years old (in the process of completing third grade) and mostly white (53.9%) non-Hispanic (85.4%). Study duration was planned to be 14 days from Visit 1 (screening and enrollment visit in which the IA evaluation questionnaire and the R-MOAS paper questionnaire was given to the subject) to Visit 2 (returning of study assessments and IA evaluation questionnaire). Study assessments included the following four measures: 1) IA evaluation questionnaire; 2) Nisonger Child Behavior Rating Form—Typical IQ; 3) Retrospective Modified Overt Aggression Scale; and 4) Caregiver Global Impression of Change.

There were two analysis types, cross-sectional and longitudinal. In the cross-sectional analyses, only a single diary per subject was employed. Such analyses included baseline concurrent validity, factor analysis and item response theory modeling. In the longitudinal analyses, more than one diary per subject was used. These analyses included change from baseline analyses used in responsiveness, test retest reliability, and anchor based MCID estimation. Psychometric analyses included the following evaluations: quality of diary completion, item distribution, inter-item correlations, factor analysis, IRT analysis, scoring, internal consistency reliability, test-retest reliability, concurrent validity, known-groups validity, and MCID estimation.

### Testing Results

1. Behavior Reporting Prevalence

A total of 14 items, or 53% of the behaviors assessed by the IA evaluation questionnaire items, were not reported with sufficient frequency to permit analysis of these items. This was determined from results pertaining to item endorsement rates, inter-item correlations, and unconditional odds of item endorsement. The endorsement rates for all items are given in Table 2. Items highlighted in gray are behaviors that are too infrequently endorsed to be analyzed. In most instances these behaviors are so extreme that the lack of reporting is logical. These include the severe injury item, the weapons item, breaking/destroying/vandalizing items, fire setting and biting items.

Weighted prevalence estimates were needed when examining the endorsement prevalence over all diaries because subjects have different numbers of diaries and we therefore had to estimate the prevalence denominated by subject-specific denominators and then pool across the n=103 subjects. Table 2 gives reporting rates for each item across columns in the following order: for qualitative interviews with n=24 caregivers, for the first diary of the validation study where n=103 subjects completed a diary, and for the weighted prevalence of endorsement across all diaries for all n=103 subjects in the validation study.

The behaviors with sufficient prevalence to be retained for the IA measure validation analyses included yelling, screaming, arguing, cursing, name calling, teasing, threatening, scratching, shoving, hair pulling, fighting, throwing, slamming, hitting self, hitting others, and kicking others. These results also provide basic epidemiologic data on the prevalence and rarity of behaviors assumed to be representative of IA in this age group but which heretofore have not been empirically assessed.

TABLE 1
Item Endorsement and Behavior Prevalence

| | | QUALITATIVE | | |
|---|---|---|---|---|
| | | PARENT | FIRST DIARY | WEIGHTED |
| ITEM | LABEL | ENDORSEMENT | ENDORSEMENT | PREVALENCE |
| 1 | YELLING | 100% | 57(55.3%) | 53.7%* |
| 2 | SCREAMING | 100% | 35(34%) | 28.5%* |
| 3 | ARGUING | 29% | 60(58.3%) | 73.6%* |
| 4 | CURSING | 50% | 8(7.8%) | 2.73% |
| 5 | NAME CALLING | 79.2% | 18(17.5%) | 18.8%* |
| 6 | TEASING | BULLY (33.3%)§ | 16(15.5%) | 8.2% |
| 7 | THREATENING | 91.7% | 9(8.7%) | 4.69% |
| 8 | SPITTING | 29.2% | 2(1.9%) | 0.25% |
| 9 | BITING | 41.7% | 1(1%) | 0.10% |
| 10 | SCRATCHING | 20.8% | 3(2.9%) | |
| 11 | SHOVING | 62.5% | 22(21.4%) | 16.61% |
| 12 | HAIR PULLING | 20.8% | 5(4.9%) | 0.27% |
| 13 | FIGHTING | 100%† | 9(8.7%) | 8.02% |
| 14 | WEAPONS | 25% | 0% | 0.11% |
| 15 | THROWING | 100% | 26(25.2%) | 15.6% |
| 16 | SLAMMING | 87.5% | 30(29.1%) | 15.9% |
| 17 | RIPPING | 29.2% | 0% | 0.65% |
| 18 | BREAKING | 62.5% | 0% | 0.47% |
| 19 | VANDALIZING | 25% | 3(2.9%) | 0.03% |
| 20 | DESTROYING | 62.5%# | 1(1%) | 0.02% |
| 21 | FIRE SETTING | 16.7% | 0% | 0.00% |
| 22 | HITTING SELF | 58.3% | 7(6.8%) | 0.82% |
| 23 | HITTING OTHERS | 100% | 33(32%) | 17.9%* |
| 24 | HITTING ANIMAL | 8.3% | 1(1%) | 0.12% |
| 25 | KICKING SELF | 0% | 2(1.9%) | 0.01% |
| 26 | KICKING OTHERS | 100% | 9(8.7%) | 3.3%* |
| 27 | KICKING ANIMAL | 8.3% | 2(1.9%) | 0.02% |
| 28 | SEVERE INJURY SELF | 0% | 0% | 0.01% |
| 29 | SEVERE INJURY OTHERS | 0% | 1(1%) | 0.00% |
| 30 | SEVERE INJURY ANIMAL | 0% | 0% | 0.00% |

*Log-Binomial failed to converge, weighted prevalence estimated by Poisson model.
§Bullying was elicited in interviews and viewed as a proxy concept supporting the broader item on teasing.
†Based on “HITTING/PUNCHING/KICKING OTHERS” in Table 4 of the qualitative report.
#Break/damage things - same percent as breaking.
Grayed out items were ineligible for analysis because of insufficient prevalence in data. These items were disqualified from further consideration in the IA evaluation questionnaire.

2. Exploratory Factor Analyses (EFAs) and Item Response Theory (IRT) Models

The number of domains/factors underlying the IA evaluation questionnaire was explored using EFA models. Model fit indices were used to determine the optimal number of factors. After determination of the optimal number of factors, item response theory models were used to evaluate the psychometric properties of the items, estimate severity rankings of behaviors, and generate empirical weights with which to construct severity weighted scores. With 16 items eligible for analysis, a total of 5 domains/factors were theoretically estimable. Therefore, the model fit of 1-5 factors to the data was explored.

Table 3 contains the model fit indices for the estimated EFA models. The χ² tests presented in Table 2 use a saturated model as the reference model for the null hypothesis. The saturated model is one which reproduces the observed inter-item tetrachoric correlations perfectly. The null hypothesis of this test is, therefore, that the estimated model fits the data no differently than the saturated, with the goal being to retain the null, thus demonstrating that an estimated model is as good as a perfect model.

Thus, for these tests, a non-significant p-value indicates failure to reject the null and the identification of an estimated model which fits as well as the perfect model. As seen in Table 2 the 1-factor model fit significantly worse than the saturated model, while the 2 and 3-factor models did not. The 4 and 5-factor models failed to converge and were considered no further. Comparative fit tests were then used to determine which of the 3 estimable models fit best relative to one another. The 2-factor model fit significantly better than the 1-factor, but the 3-factor model did not fit better than the 2-factor model. Thus, the 2-factor model was the optimal EFA solution.

TABLE 2
Tests of Absolute and Comparative Fit for EFA Models

| MODEL | x² | DF | P |
|---|---|---|---|
| 1-factor | 140.366 | 104 | 0.0101 |
| 2-factor | 84.816 | 89 | 0.60 |
| 3-factor | 70.967 | 75 | 0.61 |

| | |
|---|---|
| 4-factor | Failed to Converge |
| 5-factor | Failed to Converge |

Comparative Fit

| | | | |
|---|---|---|---|
| 1 Vs. 2 Factors | 46.706 | 15 | <0.0001 |
| 2 Vs. 3 Factors | 13.543 | 14 | 0.48 |

DF = Degrees of freedom

A series of alternative IRT models were explored in order to pick the best fitting model with which to characterize the psychometric properties of the IA evaluation questionnaire items and estimate the severity weights. Model fit for these models was compared. Models considered included a two-factor 2PL IRT model adopting the EFA factor structure, a 1PL/Rasch Bi-Factor model, and a 2PL Bi-Factor IRT model. The Bi-Factor IRT models used the EFA factor structure but added to it a general IA factor on which all items loaded. As seen in Table 3 below, model fit was best for the 2PL Bi-Factor IRT model, which had the lowest chi square and Root Mean Squared Error of Approximation (RMSEA), and highest Comparative Fit Index (CFI) and Tucker-Lewis Index (TLI).

Not only are the fit indices for the 2PL Bi-Factor IRT model better than the alternatives considered, but in absolute terms, these fit indices suggest strong fit of the model to the data, even when considering the modest sample size. However, under the bi-factor model the teasing item had a negative loading on the IA factor, suggesting that teasing was inversely related to IA. As a result, this item was eliminated from the item pool and all subsequent models were fit with a reduced 15 item pool.

TABLE 3
Model fit for Three Alternative IRT Models

| MODEL | χ² | RMSEA | LCL | UCL | CFI | TLI |
|---|---|---|---|---|---|---|
| 2 Factor | P = 0.196 | 0.035 | 0 | 0.066 | 0.935 | 0.924 |
| 2PL | | | | | | |
| Bi-Factor | P = 0.6605 | 0 | 0 | 0.047 | 1 | 1.045 |
| 2PL | | | | | | |
| Bi-Factor | P = 0.0373* | 0.051 | 0.013 | 0.076 | 0.845 | 0.841 |
| Rasch | | | | | | |

2PL stands for two parameter logistic IRT model. The 2PL estimates both a threshold and log odds ratio (referred to as a discrimination parameter in IRT parlance) for each item.
The Rasch model, also known as the 1PL, is identical to the 2PL except that it constrains the log odds ratio to equality across items and freely estimates the threshold for each item.
For the χ² statistic, the comparator model is a saturated model, thus a non-significant p-value indicates good model fit.
For the RMSEA, which quantifies model fit per DF, good fit is indicated by values of 0.05 or lower. The statistic is bounded at 0, and confidence limits with an LCL of 0 and UCL less than 0.05 indicate excellent fit.
The CFI and TLI are bounded between 0 and 1, though in rare instances the TLI can exceed 1. Values above 0.9 indicate good fit.
LCL = Lower control limit; UCL = Upper control limit

3. Overview of Internal Consistency, Reliability, Validity, and MCID Findings

The entire set of psychometric results evaluating scores, starting with internal consistency and concluding with anchor-based MCID, are summarized in Table 4. For each analysis the criterion against which it was tested is listed, consolidated observed results are given, and whether the criterion was satisfied is indicated. All but three analyses (internal consistency for unweighted scores, distribution-based MCID for change and anchor-based MCID) satisfied their criterion.

The internal consistency estimate for the unweighted score likely fell below the criterion because of measurement-error-based attenuation. In the case of both distribution-based MCID for change and anchor-based MCID, the non-interventional nature of the study design explained the failure to meet the criterion of success. Therefore, compelling evidence supporting the psychometric validation of the IA evaluation questionnaire was found in this study.

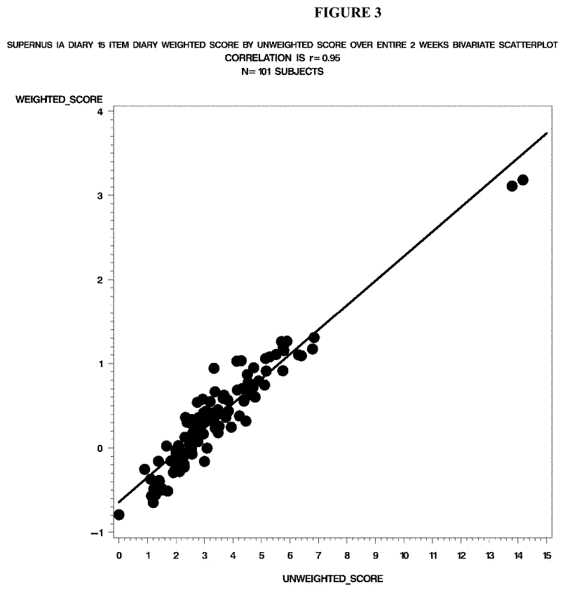

In this study, weighted and unweighted scores were correlated approximately 0.95 (FIG. 3). While weighted scores are used to generate scores that conform to the model structures detected during analysis, such a high correlation with the unweighted score demonstrates that the two scores share roughly 90% explained variance. With such a high level of shared variance, the unweighted scores, which are much easier to calculate and whose frequency interpretation is clear and intuitive, are logical candidates from which to construct endpoints.

There was strong correspondence in reliability and validity findings for the weighted and unweighted scores. For virtually every statistic estimated in a common metric the results for weighted and unweighted scores were within rounding error. This suggests that the unweighted scores are a good approximation to the gold standard weighted scores in this context. In the case where analyses necessitated score-dependent models producing estimates in different metrics the same comparison was not possible. But even for those analyses, the outcome of analysis was identical, with each score succeeding or failing to achieve their criterion in unison.

Consequently, the unweighted score appears to be a psychometrically fit measure of IA from which endpoints can be constructed.

TABLE 4
Analysis Results Summary

| | | Weighted Scores | | Unweighted Scores | |
|---|---|---|---|---|---|
| Analysis | Criterion | Estimate | Succeed | Estimate | Succeed |
| Internal Consistency | 0.8≤ | 0.86 | Yes | 0.73 | No |
| TRT | 0.7 - 0.8≤ | 0.80 | Yes | 0.77 | Yes |
| Concurrent Validity | 0.4≤ | 0.58 | Yes | 0.49 | Yes |
| R-MOAS 1 | | | | | |
| Concurrent Validity | 0.4≤ | 0.63 | Yes | 0.62 | Yes |
| R-MOAS 2 | | | | | |
| Concurrent Validity | 0.4≤ | 0.44 | Yes | 0.41 | Yes |
| NCBRF TIQ | | | | | |
| D-TOTAL | | | | | |
| Known Groups | Strong Positive | 0.7 SD Higher | Yes | Rate of behavior | Yes |
| Validity R-MOAS 1 | Effect | | | frequency 111% | |
| higher | | | | | |
| Known Groups | Strong Positive | 0.44 SD Higher | Yes | Rate of behavior | Yes |
| Validity R-MOAS 2 | Effect | | | frequency 45% | |
| higher | | | | | |
| Known Groups | Strong Positive | 0.35 SD Higher | Yes | Rate of behavior | Yes |
| Validity NCBRF TIQ | Effect | | | frequency 51% | |
| D-TOTAL | | | | higher | |
| Distribution-Based | Mean Exceeding | SEM: | Yes | SEM: | Yes |
| MCID | SEM | W1 = 0.25 | | W1 = 1.09 | |
| | | W2 = 0.27 | | W2 = 1.14 | |
| | | ALL = 0.24 | | ALL = 1.07 | |
| | | AVERAGE | | AVERAGE | |
| | | SCORES: | | SCORES: | |
| | | W1 = 0.34 | | W1 = 3.49 | |
| | | W2 = 0.38 | | W2 = 3.43 | |
| | | ALL = 0.36 | | ALL = 3.45 | |
| Distribution-based | Negative Significant | Never | No | Never | No |
| MCID of Change | Change | | | | |
| Anchor-based MCID | Significant | Logical ordering | No | Logical ordering | No |
| with CGIC | Differences in with | but never | | but never | |
| | order of magnitude | statistically | | statistically | |
| | matching CGIC | significant. | | significant. | |
| | severity group. | | | | |

TRT = Test retest

### Example 2: Molindone Clinical Trial with Diary Monitoring

A randomized, double-blind, placebo-controlled study is conducted to evaluate the efficacy and safety of molindone hydrochloride extended-release tablets for the treatment of impulsive aggression in pediatric patients with attention deficit/hyperactivity disorder (ADHD) in conjunction with standard ADHD treatment. The study is a double-blind, placebo controlled, 3-arm, randomized (1:1:1), parallel group study. Approximately 378 subjects aged from 6 to 12 years are screened to achieve 291 subjects randomized (or 97 subjects per treatment arm).

Subjects meeting the following criteria are included in the study:

Otherwise healthy male or female subjects, age 6 to 12 years at the time of screening with a primary diagnosis of ADHD and currently receiving an optimized FDA-approved ADHD medication. Subjects are confirmed for impulsive aggression using a validated scoring method—R-MOAS and/or the Vitiello Aggression Scale.

The study is conducted for 10-12 weeks in phases, as noted below:
Pre-treatment phase: 4-6 weeks
   Screening period: 2-4 weeks
   Baseline period: 2 weeks
Treatment phase: 5 weeks
   Titration period: 2 weeks
   Maintenance period: 3 weeks
Conversion/taper phase: 1 week

Subjects are divided into three groups—Group 1 receiving placebo, Group 2 receiving 18 mg of molindone, and Group 3 receiving 36 mg of molindone. Molindone is administered in the form of molindone hydrochloride extended-release tablet dosage forms of 3 mg to 9 mg with matching placebo tablets. Treatment is administered orally, twice a day with food. Subjects are force-titrated over a period of 2 weeks to their final randomized dose.

Subjects are monitored using data inputted into the IA diary. Impulsive aggressive behavior frequencies are calculated based on unweighted scores—the sum of the total impulsive aggressive behaviors observed (of the 15 validated in Example 1)—over 7 days in the maintenance period versus the baseline period over the number of days with complete IA diary input.

Pharmacokinetic information is obtained from each subject by analysis of five blood samples divided between two visits. The concentration of molindone, amphetamine, methylphenidate, guanfacine, clonidine, and atomoxetine in the sample plasma is determined using validated chromatographic methods.

Efficacy is calculated as a percentage change from baseline—(M-B)/B, where B is the baseline period frequency and M is the maintenance period frequency, multiplied by 100 to yield a percentage.

Statistical significance is assessed according to the Wilcoxon rank-sum test with a 2-sided significance level, where alpha is 0.05. Subjects who received at least one dose of study drug are assessed for safety; subjects who also had a baseline and at least 1 valid post-randomization assessment of IA behavior frequency based on the diary are assessed to validate intent-to-treat. Further analysis of each administered protocol is based on those subjects assessed for intent-to-treat, with 80% diary completion and no major protocol deviations.

All subjects are assessed for pharmacokinetic information. A population pharmacokinetic model is developed for molindone using non-linear mixed-effects modeling (NOMEM). Covariates are included through a systematic process and include age, gender, body size, treatment, organ function, and ADHD medication.

1. A method of treating a patient having impulsive aggression (IA) by administering an impulsive aggression intervention, comprising:
(a) monitoring the patient for one or more aggressive episodes by:
providing the patient or a caregiver of the patient with a questionnaire to determine a pre-intervention baseline cumulative score of the patient,
initiating an impulsive aggression intervention treatment, and
providing the patient or the caregiver with the questionnaire to determine a post-intervention cumulative score of the patient; and either
(b) continuing to administer the impulsive aggression intervention treatment if the post-intervention cumulative score is lower than the pre-intervention baseline cumulative score; or
(c) varying the impulsive aggression intervention treatment if the post-intervention cumulative score is not lower than the pre-intervention baseline cumulative score.

2-7. (canceled)

8. The method of claim 1 wherein the pre-intervention baseline cumulative score is calculated by monitoring the patient having impulsive aggression for one or more aggressive episodes prior to administration of the impulsive aggression intervention treatment.

9. The method of claim 1, wherein the questionnaire comprises a diary used by the patient or the caregiver to report each of the one or more aggressive episodes as observed.

10. The method of claim 9, wherein the questionnaire further prompts the caregiver to review each of the one or more aggressive episodes reported for a day and enter any aggressive episodes that were not previously reported.

11. The method of claim 1, wherein each of the one or more aggressive episodes is characterized by one or more behaviors from a group of behaviors comprising yelling, screaming, threatening, scratching, throwing, slamming, hitting self, arguing, cursing, name calling, shoving, hair pulling, fighting, hitting others, and kicking others; and wherein each of the one or more behaviors are coded as observed (coded 1) or not observed (coded 0) based on answers from the patient or the caregiver.

12. The method of claim 11, wherein a frequency of one or more behaviors over a fixed period of time is calculated to generate a score.

13. The method of claim 12, wherein the fixed period of time is a day, a week, a month, or a year.

14. The method of claim 12, wherein the pre-intervention baseline cumulative score is calculated based on two scores, each representing the frequency of the one or more behaviors provided in the questionnaire; and wherein the post-intervention cumulative score is calculated based on two scores, each representing the frequency of the one or more behaviors provided in the questionnaire.

15. The method of claim 14, wherein the pre-intervention baseline cumulative first score is calculated based on the frequency of a group of behaviors consisting of: yelling, screaming, threatening, scratching, throwing, slamming, and hitting self; and wherein the pre-intervention baseline cumulative second score is calculated based on the frequency of a group of behaviors consisting of: arguing, cursing, name calling, shoving, hair pulling, fighting, hitting others, and kicking others.

16. The method of claim 1, wherein the pre-intervention baseline cumulative score and the post-intervention cumulative score is calculated as a weighted score.

17. The method of claim 1, wherein the pre-intervention baseline cumulative score and the post-intervention cumulative score is calculated as an unweighted score.

18. The method of claim 1, wherein the impulsive aggression intervention treatment administered is selected from behavioral therapy or administration of molindone.

19. The method of claim 18, wherein the impulsive aggression intervention treatment is administration of molindone according to a dose or dose regimen.

20. The method of claim 19, wherein the dose of molindone is between about 3 mg and about 36 mg.

21. The method of claim 20, wherein the dose of molindone is about 18 mg.

22. The method of claim 20, wherein the dose of molindone is about 36 mg.

23. The method of claim 19, wherein continued administration of the impulsive aggression intervention comprises maintaining, increasing, or lowering the dose of molindone.

24. The method of claim 1, wherein the patient is a child or an adolescent.

25. The method of claim 24, wherein the child is between the ages of about 6 to about 12.

26. The method of claim 24, wherein the adolescent is between the ages of about 12 to about 17.

27. The method of claim 1 wherein the patient having impulsive aggression has impulsive aggression associated with a primary condition.

28. The method of claim 27, wherein the primary condition is attention deficit hyperactivity disorder (ADHD), bipolar disorder, autism, Tourette's syndrome, or post-traumatic stress disorder (PTSD).

29-94. (canceled)

95. The method of claim 1, wherein the impulsive aggression intervention treatment initiated is a secondary treatment.

96. The method of claim 13, wherein the fixed period of time is a week or seven days.

97. The method of claim 14, wherein the post-intervention cumulative first score is calculated based on the frequency of a group of behaviors consisting of: yelling, screaming, threatening, scratching, throwing, slamming, and hitting self; and wherein the post-intervention cumulative second score is calculated based on the frequency of a group of behaviors consisting of: arguing, cursing, name calling, shoving, hair pulling, fighting, hitting others, and kicking others.

98. The method of claim 19, wherein the dose of molindone is between about 12 mg and about 36 mg.

99. The method of claim 20, wherein the dose of molindone is about 12 mg.
